(19) United States
(12) Patent Application Publication
(10) Pub. No.: US 2021/0069443 A1
(43) Pub. Date: Mar. 11, 2021

(54) MEDICAL VENTILATOR PROTECTED BY AN EXOSKELETON STRUCTURE

(71) Applicant: Air Liquide Medical Systems, Antony (FR)

(72) Inventors: Pauline GIARD, Saint Germain le Vasson (FR); Sofia ANDRE DIAS, Antony (FR); Damien GERMANI, Châtillon (FR); Catherine HARANT, Plaisir (FR); Nicolas LEBATTEUR, Bretigny sur Orge (FR); Julien LOPEZ, Châtillon (FR); Leslie RUSSO, Sivry-Courtry (FR)

(21) Appl. No.: 16/915,489

(22) Filed: Jun. 29, 2020

(30) Foreign Application Priority Data
Sep. 11, 2019 (FR) 1909987

(51) Int. Cl. A61M 16/06; A61M 16/00

(52) U.S. Cl. CPC A61M 16/0683; A61M 16/0066; A61M 2205/583; A61M 2209/084; A61M 16/0003

(57) ABSTRACT

The invention relates to a medical ventilator (1) comprising an external housing (2), and a rigid exoskeleton structure (3) arranged around the external housing (2) and rigidly connected thereto. The rigid exoskeleton structure (3) comprises a plurality of elongate elements (4) defining a volume (5) in which the external housing (2) is housed, the greater part of the rigid exoskeleton structure (3) being spaced (7) from said external housing (2) when said external housing (2) is housed in the volume (5). A carrying handle (9) is also provided, allowing a user to manually grasp the exoskeleton structure (3) and transport the assembly (1, 3) composed of ventilator and exoskeleton structure.

### CROSS REFERENCE TO RELATED APPLICATION

This application claims the benefit of priority under 35 U.S.C. § 119 (a) and (b) to French Patent Application No. 1909987, filed Sep. 11, 2019, the entire contents of which are incorporated herein by reference.

### BACKGROUND

The invention relates to a medical ventilator protected by an exoskeleton structure which is arranged around the external housing of the medical ventilator in such a way as to protect the latter against impacts or the like, in particular a ventilator suitable for emergency interventions and for transportation.

In order to assist certain persons or patients with their respiratory function, use is normally made of a respiratory assistance apparatus, also called a medical ventilator, comprising a motorized micro-blower delivering a respiratory gas, such as air or oxygen-enriched air, at a non-zero flow rate and/or at a pressure higher than atmospheric pressure (>1 atm).

The motorized micro-blower, also called a compressor or turbine, aspirates ambient air and delivers it at a given pressure to the patient. The aspiration of the air by the micro-blower, during the functioning of its motor, is effected by virtue of one or more bladed wheels which are arranged on a rotary shaft driven in rotation by an electric motor, the one or more bladed wheels being movable in rotation in the internal compartment of the volute of the micro-blower. The air can be oxygen-enriched air, that is to say it can have additional oxygen added to it.

The documents EP-A-2165078, EP-A-2102504, WO-A-2012/139681, US-A-2008/0304986 and WO-A-2013/020167 describe such medical ventilators.

Some ventilators, called emergency transport ventilators, are intended to be transported in the field by civilian or military first responders and to be used during emergency interventions outdoors, for example in the event of accidents, natural disasters, conflicts, chemical or biological attacks or the like.

Thus, an emergency transport ventilator must be able to be used in a land ambulance or air ambulance, i.e. a helicopter for example, placed on a wall-mounted recharging point or fastened to a stretcher or to a wall, or placed on a surface, in particular the floor. Similarly, it must also be able to be used outdoors, including in difficult conditions, especially in the presence of sand, snow or mud, in a salty environment, and at extreme temperatures.

Emergency transport ventilators are thus subject to severe stresses during use, in particular to impacts, droppages, vibrations or other forms of external aggression.

To protect them, a transportation bag is commonly used which is made of flexible material (e.g. woven polymer fabric, etc.) and in which the medical ventilator is placed and protected. However, it will be readily appreciated that this solution is not ideal, since the transportation bags are not able to adequately protect the ventilator against heavy impacts, droppages or other forms of aggression. Moreover, the presence of the bag around the ventilator complicates access to the attachment points, the connector elements, the HMI (i.e. the human-machine interface) or other elements and can also make it difficult to fasten the ventilator to certain wall supports, particularly in emergency vehicles (e.g. ambulances, helicopters, etc.) and is detrimental to the cooling of the medical ventilator.

Moreover, FR-A-3076223 proposes a support frame composed of tubes for carrying a medical ventilator, a mask compartment, a gas cylinder and other elements. The whole assembly is transported via two lateral arches situated at the ends of the frame.

WO-A-2014/145253 discloses an emergency transport device for children. There too, the device is transported via two lateral arches.

Such assemblies are heavy and cumbersome and provide only relative and incomplete protection.

The problem is to be able to efficiently protect a medical emergency transport ventilator against severe stresses during use, in particular impacts, droppages, vibrations or other forms of external aggression, to which it may be exposed during its transportation or use, particularly during emergency interventions performed outdoors, for example in the event of accidents, natural disasters, conflicts or the like, while at the same time permitting easy manipulation of the ventilator and without impeding or preventing access to the attachment points, the connector elements, the HMI or other elements of the ventilator. Moreover, the medical ventilator must be easy to transport.

### SUMMARY

The solution according to the invention is therefore a medical ventilator, in particular a medical emergency transport ventilator, comprising an external housing and a rigid exoskeleton structure arranged around said external housing and rigidly connected to said external housing.

According to the embodiment considered, the medical ventilator or respiratory assistance apparatus of the invention can comprise one or more of the following features:
the exoskeleton structure encloses the entire external housing of the ventilator, that is to say it forms a rigid protective fairing that envelops/surrounds all of (and exclusively) the external housing of the ventilator.
the rigid exoskeleton structure comprises a plurality of elongate elements defining a volume in which the external housing of the ventilator is housed.
the rigid exoskeleton structure comprises at least four elongate elements.
the elongate elements are spaced apart from one another.
the rigid exoskeleton structure comprises a base.
the elongate elements are rigidly connected to the base of the exoskeleton structure.
the base of the exoskeleton structure curves outwards.
the base of the exoskeleton structure comprises a slightly curved plate.
the base of the exoskeleton structure comprises a bearing surface arranged on the outer face of the base, the bearing surface preferably curving outwards.
the bearing surface of the base of the exoskeleton structure rests on and is in contact with the ground (or any other surface) when the assembly composed of ventilator and exoskeleton structure is placed on the ground in an “upright” position, so as to avoid the ventilator coming into direct contact with the ground and thereby protect the ventilator from possible damage when the ground is covered with sand, mud, snow or water, etc.
the elongate elements preferably have the general shape of a band or tape.
the greater part of the exoskeleton structure is spaced from the external housing when the external housing is housed in the volume of the exoskeleton structure, that is to say except for the base of the exoskeleton structure, in which region the external housing of the ventilator is fixed.
the greater part of the exoskeleton structure is spaced from the external housing by a distance of less than 10 cm, preferably less than 5 cm, when the external housing is housed in the volume of the exoskeleton structure.
the external housing positions itself on the base of the rigid exoskeleton structure in such a way that the external housing is not in direct contact with the surface, such as the ground, on which the ventilator is placed.
the external housing fixes itself detachably, i.e. removably, to the base of the exoskeleton structure.
the external housing is fixed to the base of the exoskeleton structure.
the external housing of the ventilator comprises a front face or façade having a display screen and/or a HMI.
the exoskeleton structure, in particular the bearing surface of the base of the exoskeleton structure, is configured in such a way that the ventilator is slightly inclined, that is to say not strictly vertical, when it is placed in an “upright” position on a surface, in particular the ground, so as to improve the user's view of the HMI.
the external housing comprises a rear face situated opposite the front face.
the external housing comprises two lateral faces arranged between the front face and the rear face, that is to say on either side of the external housing, i.e. on the right and left.
the front face, the rear face and the two lateral faces are separated by four corner regions forming a join between the faces, that is to say a corner region is situated at the limit or junction between two successive faces, and in particular the corner regions are ridges or the like.
the elongate elements of the exoskeleton structure are positioned, wholly or partly, substantially opposite, i.e. facing and at a short distance from, the corner regions of the ventilator, that is to say the ridge regions separating the faces of the ventilator external housing.
the general shape of the ventilator external housing is approximately parallelepipedal.
the elongate elements comprise a first end rigidly connected to the base.
the elongate elements are arranged substantially vertically from the base when the ventilator is in an “upright” position, that is to say when it is resting on the base of the exoskeleton structure.
the elongate elements meet and join, via a second end, at a joining zone of the exoskeleton structure above the ventilator.
a carrying handle is provided at the joining zone of the exoskeleton structure.
each elongate element comprises a plurality of successive portions or segments forming an overall structure that is non-rectilinear, that is to say the segments are separated from one another by bends (i.e. corners).
each elongate element comprises a plurality of successive portions (or segments), including a first portion having the first end, a second portion having the second end, and an intermediate portion situated between the first and second ends.
the first and second portions and the intermediate portion are rectilinear or approximately rectilinear.
the first portion forms a first angle A of greater than 90° with the intermediate portion.
the second portion forms a second angle B of greater than 90° with the intermediate portion.
the angles A and B are between 110° and 150°, preferably of the order of 120 to 135°.
the angles A and B are equal or different.
the exoskeleton structure comprises four elongate elements.
the exoskeleton structure comprises four elongate elements which are substantially vertical when the ventilator is in an “upright” position, that is to say when the base of the rigid exoskeleton structure is resting on a surface such as the ground.
the four elongate elements each comprise an axis XX, the axes XX of the four elongate elements being (approximately) parallel to one another.
the axes XX of the four elongate elements are carried by their intermediate portion.
the exoskeleton structure is moreover configured such that the ventilator is slightly inclined (with respect to the surface in question) when it rests in a “recumbent” position on a surface, in particular a horizontal surface, such as the ground, and such that the screen of the ventilator is then located at the top and in a horizontal position or in a position slightly inclined with respect to the horizontal position.
the exoskeleton structure comprises two “rear” elongate elements situated opposite the rear face of the external housing of the ventilator, that is to say the face opposite the front façade of the external housing supporting the screen.
the two “rear” elongate elements comprise coplanar bearing zones, that is to say bearing zones situated in the same plane, preferably situated in their intermediate portion of axis XX, so as to be able to place the assembly composed of ventilator and exoskeleton structure in a “recumbent” position on a surface, such as the ground, such that the screen of the ventilator is located at the top and is (approximately) horizontal or slightly inclined.
the “recumbent” and “upright” positions are stable positions of the assembly composed of ventilator and exoskeleton structure. In other words, the exoskeleton structure is configured such that the ventilator can offer a plurality of positions ensuring good stability, in particular the positions designated “recumbent” and “upright”.
the elongate elements join in pairs in the joining zone.
the elongate elements join in pairs on either side of the exoskeleton structure, forming two Y-shaped or V-shaped joining structures.
the two Y-shaped or V-shaped joining structures are situated on either side of the exoskeleton structure and are connected to each other by the central region comprising the gripping handle, and the central region is preferably elongate in shape.
the central region of the joining zone forms the carrying handle.
the carrying handle is dimensioned to allow a user, such as a first responder, to grasp it manually and lift and easily transport the assembly composed of ventilator and exoskeleton structure by hand.
the carrying handle is sandwiched between the two Y-shaped or V-shaped joining structures.
the exoskeleton structure moreover comprises additional strengthening elements, preferably one or more intermediate wall elements joining two elongate elements to each other, for example half way between the first and second ends of said elongate elements, so as to fix them firmly to each other and thereby further stiffen the exoskeleton structure.
the exoskeleton structure comprises four elongate elements forming pairs, in particular a pair of front elements and a pair of rear elements.
the two elongate elements of one pair are symmetrical with respect to each other and are situated at an equal distance from the external housing of the ventilator.
the exoskeleton structure forms a rigid cage which (almost) completely surrounds the external housing of the ventilator, that is to say which preferably encloses all the faces of the ventilator external housing.
the exoskeleton structure comprises elongate elements extending upwards from the base supporting the ventilator, said elongate elements completely surrounding said ventilator but being arranged at a distance from the latter, i.e. from its external housing, and joining each other above the ventilator at a joining zone forming the carrying handle, when the ventilator is in the upright position.
the external housing contains a micro-blower.
the micro-blower is equipped with an electric motor rotating a shaft, also called a rotary shaft, supporting one or more bladed wheels.
the micro-blower comprises a volute delimiting a wheel compartment in which a bladed wheel is arranged.
the volute comprises a gas outlet passage in fluidic communication with the wheel compartment, in order to extract from said wheel compartment a gas flow generated by the bladed wheel, when said bladed wheel is driven in rotation by the shaft.
the external housing forms a casing or the like.
the external housing comprises rigid walls.
the front wall forming all or part of the front façade comprises the display screen.
the external housing is made of polymer.
the external housing also comprises control means configured to control the functioning or shut-down of the electric motor, that is to say the rotations and the discontinuation of rotation (i.e. braking or deceleration) of the wheel of the micro-blower.
the control means comprise at least one microprocessor, preferably at least one microcontroller.
the control means comprise at least one electronic board comprising said at least one microprocessor.
the microprocessor uses one or more algorithms.
the external housing comprises electrical supply means, in particular one or more rechargeable batteries.
the control means are supplied with electric current by the electrical supply means.
it comprises a HMI, that is to say a human-machine interface.
the HMI comprises means for selections or settings, such as buttons or keys.
the display screen is a touch screen and comprises, forms or is part of the HMI.
the electric motor comprises electric cables or wires for connecting it electrically to a source of electric current.
during its operation, the electric motor drives the bladed wheel in rotation at a speed of as high as 70,000 rpm, typically as high as 30,000 or 40,000 rpm.
the electric motor is of the brushless type.

### BRIEF DESCRIPTION OF THE DRAWINGS

The invention will now be better understood by virtue of the following detailed description, which is provided by way of a non-limiting illustration, and with reference to the appended figures, in which:

FIG. 1 is a first view, substantially from the front, of an embodiment of a medical ventilator equipped with an exoskeleton structure according to the invention, shown in an “upright” position,

FIG. 2 is an embodiment of an exoskeleton structure for a medical ventilator according to the invention,

FIG. 3 is a view of the assembly of the exoskeleton structure with the handle on the external housing of a medical ventilator according to the invention, in an intermediate position, the exoskeleton structure with handle not being fixed to the external housing,

FIG. 4 is a view showing the ventilator from FIG. 1 in an inclined position,

FIG. 5 is substantially a side view of the ventilator from FIG. 1, shown in a “recumbent” position, and

FIG. 6 is a plan view of the rear part of the ventilator from FIG. 1.

### DETAILED DESCRIPTION OF PREFERRED EMBODIMENTS

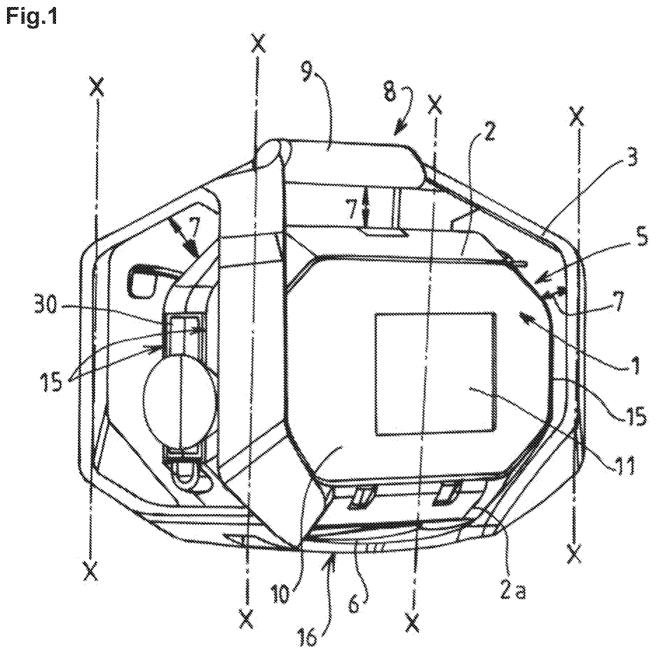

FIG. 1 is a first view, substantially from the front, of an embodiment of a medical ventilator 1, typically for emergency transport, equipped with an exoskeleton structure 3 according to the invention arranged around the external housing 2 or casing of the ventilator 1. The exoskeleton structure 3 protects solely the ventilator 1, that is to say it is not aimed at protecting another appliance of any additional device associated with the ventilator 1.

The ventilator 1 is shown in an “upright” position, that is to say with its front façade 10, having the display screen 11, substantially vertical, and with its bottom 2a situated at the surface (not shown) on which it is placed, for example the ground.

The display screen 11 makes it possible to view information, data, alarms, monitoring curves, etc. It can be a black and white screen or a colour screen. It can be a touch screen and can have or form a HMI, that is to say it can have selection buttons for performing adjustments, making choices from menus, activating functions, acknowledging alarms, selecting ventilation modes or other parameters.

The external housing 2 of the ventilator 1 forms a rigid casing, for example made of polymer.

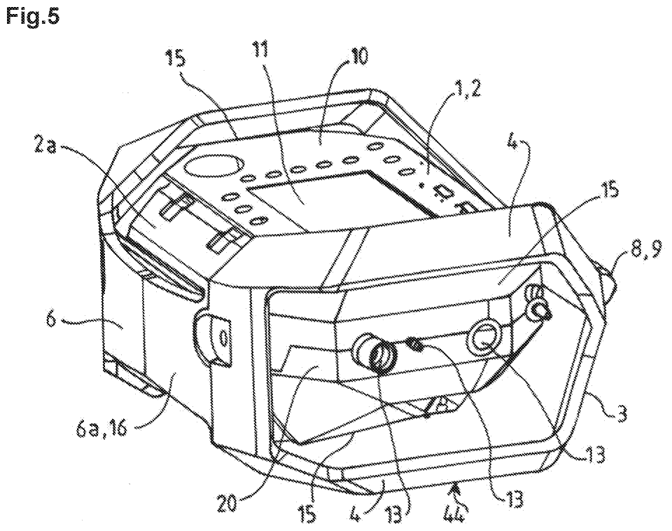

Traditionally, the external housing 2 of the ventilator 1 has various elements and components, such as sockets, connectors or attachment points 13 (cf. FIG. 5) to which hoses or other flexible conduits will be attached mechanically and fluidically, in particular a patient circuit connecting the ventilator 1 to the patient and serving to deliver the gas to the latter, which traditionally comprises at least one flexible conduit and terminates with a respiratory interface, for example a breathing mask or similar.

The external housing 2 of the ventilator 1 also encloses control means, such as an electronic board with microprocessor implementing one or more algorithms, which are configured to govern the function or the shut-down of the electric motor of the micro-blower, that is to say the rotations and the interruptions in rotation (i.e. braking or deceleration).

Preferably, the electric motor is of the brushless type and, during its operation, it drives the bladed wheel in rotation at a speed of as high as 70,000 rpm, typically as high as 30,000 or 40,000 rpm.

Means for supplying electric current are also provided, in particular one or more rechargeable batteries supplying electric power to the control means, the display screen, the HMI, the motor of the micro-blower or other components of the ventilator that require electric current in order to function, especially via cables or electrical connection wires.

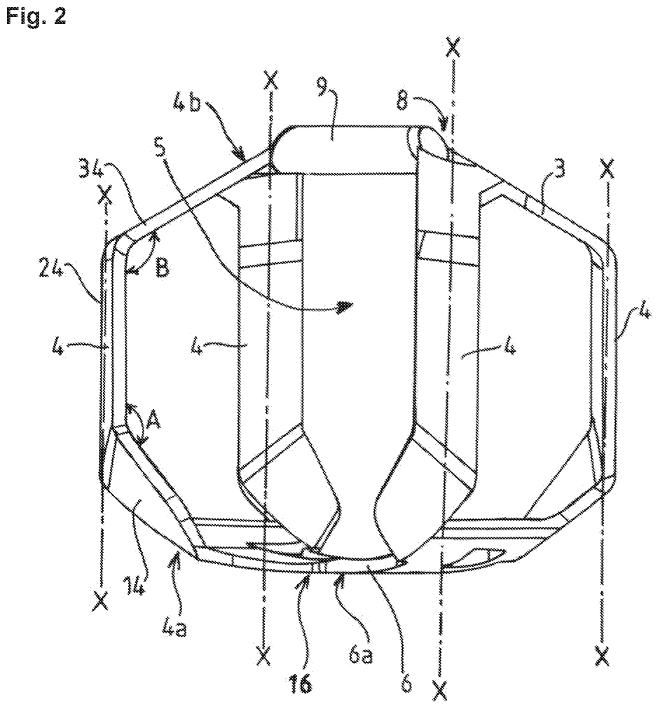

According to the present invention, the medical ventilator 1 comprises a rigid exoskeleton structure 3 which is arranged around said external housing 2 and is rigidly connected thereto. An embodiment of the rigid exoskeleton structure 3 is illustrated in FIG. 2.

This rigid exoskeleton structure 3 can be formed from one or more rigid materials, especially polymer, metal or metal alloy, e.g. an aluminium alloy, steel or Zamac, composite material or similar. The rigid exoskeleton structure 3 as a whole can be formed in one piece or can be formed from a plurality of pieces or sub-units that are assembled, for example by screwing or similar.

As will be seen in FIG. 2, the rigid exoskeleton structure 3 comprises a plurality of elongate elements 4, also called arms, which are spaced apart from one another and are configured to define or delimit a volume 5, such as a cage or rigid cowl, in which the external housing 2 of the ventilator will be housed.

In this embodiment, the rigid exoskeleton structure 3 comprises a base 6, or platform, and four elongate elements 4 rigidly connected to said base 6. The elongate elements 4 can be formed or fashioned in one piece with the base 6, for example by injection moulding or similar, or can be fixed thereto, directly or indirectly, for example via an intermediate piece. Preferably, the base 6 and the elongate elements 4 are formed in one piece.

Here, the base 6 is in the shape of a slightly curved plate; however, it can have another shape and/or can be open-worked. The provision of a base 6 that is slightly curved, that is to say bulges outwards, on the bottom of the exoskeleton structure 3 is advantageous since, in the event of the assembly composed of ventilator 1 and exoskeleton structure 3 being dropped, it ensures that the impact energy is not transmitted integrally to the apparatus 1 but is partially transformed into kinetic energy that is absorbed by the exoskeleton structure 3.

This base 6 comprises, on its outer face 6a, a bearing surface 16 which preferably bulges outwards and rests on the surface, for example the ground, on which the assembly composed of ventilator 1 and exoskeleton structure 3 is placed in the “upright” position, as is illustrated in FIGS. 1 to 4. It is thereby possible to avoid the ventilator 1 coming directly into contact with the ground, thus protecting it from possible damage when the ground is covered with sand, mud, snow, water, etc.

For their part, the elongate elements 4 here have the general shape of a band or tape, although here too they could have another shape, for example a tubular shape, in particular cylindrical or similar. It is also possible to provide more than four elongate elements 4, and/or these elements can be divided longitudinally. In addition, the elongate elements 4 do not necessarily all have the same shape and/or the same dimensions.

As is illustrated, the elongate elements 4 also project upwards and meet above the ventilator 1, that is to say in a joining zone 8 situated above the summit 2b of the external housing 2, that is to say opposite the bottom surface 2a.

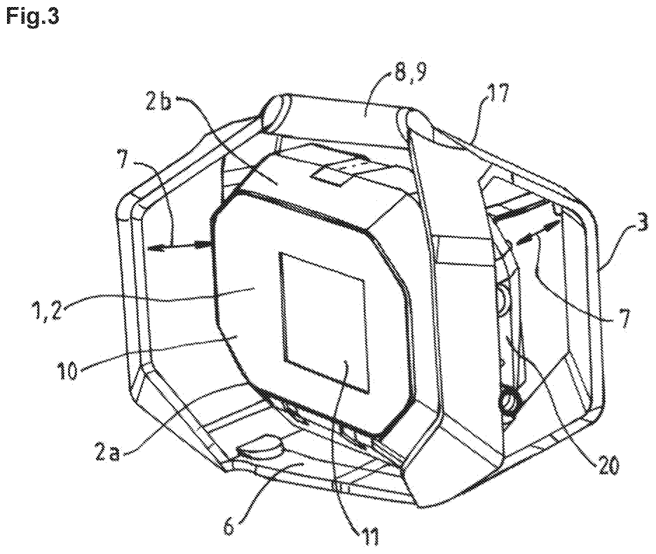

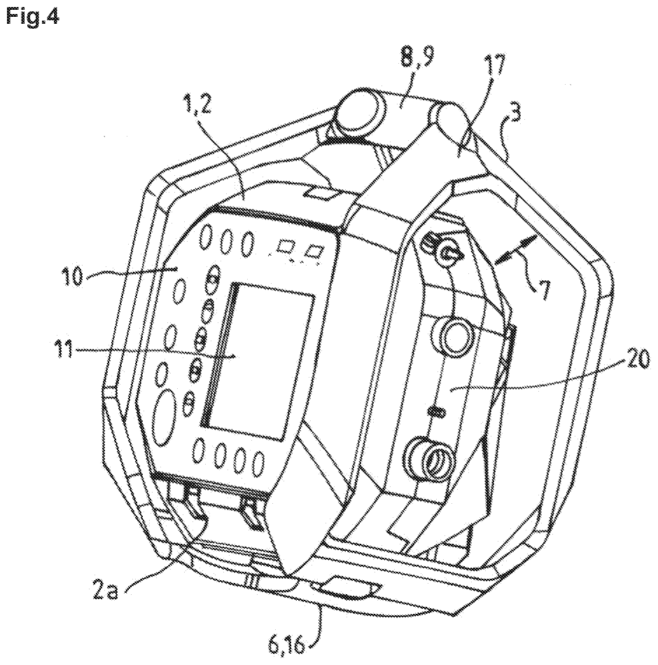

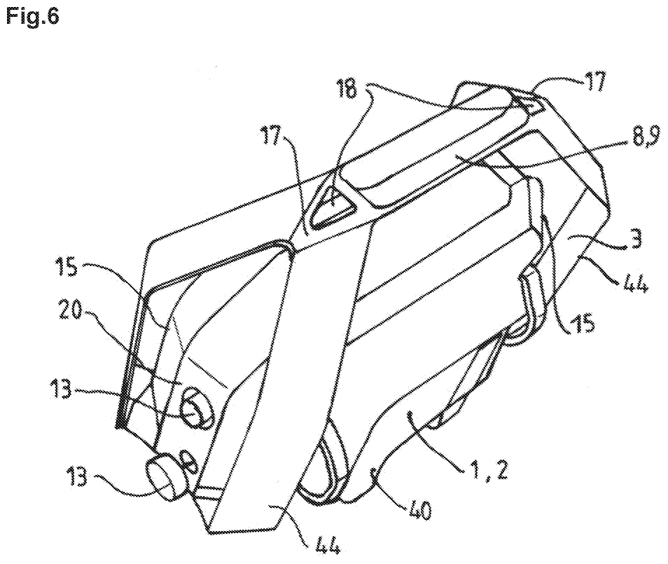

More precisely, as will be seen in FIG. 3, FIG. 4 and FIG. 6, in the joining zone 8 the elongate elements 4 join each other in pairs, on either side of the exoskeleton structure 3, forming two Y-shaped or V-shaped joining structures 17 which are themselves also situated on either side of the exoskeleton structure 3 and are connected to each other by a central region, preferably of elongate shape.

Advantageously, the central region of the joining zone 8 forms a carrying handle 9 dimensioned to allow a user, such as a first responder, to grasp it manually, that is to say by hand, and lift and easily transport the assembly composed of ventilator 1 and exoskeleton structure 3. In other words, the carrying handle 9 is sandwiched between the two Y-shaped or V-shaped joining structures 17.

The carrying handle 9 has a cylindrical shape, for example, and is arranged horizontally when the ventilator 1 is placed in the “upright” position on a surface, for example the ground, as is illustrated for example in FIG. 1 and FIG. 3. The carrying handle 9 can be formed or fashioned in one piece with the rest of the exoskeleton structure 3 or can be fixed, directly or indirectly, to the elongate elements 4. It can be formed from one or more pieces. It can be covered with a flexible material that has a soft feel, for example an elastomer or silicone, so as to improve the sensation for the user who picks it up, preferably a soft material with a Shore hardness 0 to 50.

Preferably, the carrying handle 9 is arranged substantially parallel to the plate forming all or part of the base 6, as is shown schematically in FIG. 2. However, it should be noted that the handle 9 is optional, that is to say it is not always essential and can thus be omitted in some cases of use. In addition, according to the chosen embodiment, it is also possible to provide a plurality of carrying handles 9 arranged on the rigid exoskeleton structure 3.

It should be emphasized that a user can also take hold of the assembly and transport it by gripping one of the elongate elements 4, that is to say instead of gripping the carrying handle 9.

More generally, the rigid exoskeleton structure 3 has one or more planes of symmetry, in particular the right-hand part of the exoskeleton structure 3 illustrated in FIG. 2 is (approximately) symmetrical with its left-hand part, and/or its front part is also (approximately) symmetrical with its rear part.

Moreover, when the external housing 2 of the ventilator 1 is housed in the volume 5, which is defined or delimited between the elongate elements 4, the base 6 and the joining zone 8 including the handle 9, the greater part of the rigid exoskeleton structure 3 is spaced 7 from the external housing 2, that is to say that the elongate elements 4 are not in direct contact with the surface/peripheral wall of the external housing 2, except at the base 6, but are spaced therefrom, for example by a few mm to a few cm, such that a mechanical impact or droppage leading to a deformation of the exoskeleton structure 3, that is to say of one or more elongate elements 4, can be absorbed by the exoskeleton structure 3 and cannot transfer to the peripheral wall of the external housing 2 of the ventilator 1, thereby protecting the latter effectively.

More precisely, the external housing 2 of the ventilator 1 comes to rest here on the base 6 of the rigid exoskeleton structure 3 and is fixed via its bottom surface 2a to said base 6, by which means it is also possible to insulate the ventilator 1 from the ground and thus avoid direct contact thereof with media that could damage it, such as sand, soil, gravel, snow, dampness, etc., since it is then the exoskeleton structure 3 that is in contact with the ground via its base 6.

Moreover, fixing the rigid exoskeleton structure 3 to the external housing 2 of the ventilator in its lower part, that is to say at its bottom surface 2a, spacing 7 the greater part of the rigid exoskeleton structure 3 from the external housing 2, makes it possible to leave room for a deformation of the rigid exoskeleton structure 3 in the event of a droppage/impact that acts on the elongate elements 4 or other parts of the exoskeleton, but without affecting the external housing 2 of the ventilator 1 itself and thus without compromising the correct function of the ventilator 1. Of course, the exoskeleton structure 3 can also be rigidly connected to the external housing 2 of the ventilator 1 at one or more other sites, that is to say other than at its bottom surface 2a, according to the chosen embodiment.

More precisely, the elongate elements 4 each comprise a first end 4a rigidly connected to the base 6 and a second end 4b rigidly connected to the carrying handle 9, that is to say at the joining zone 8, which is to say that the elongate elements 4 are arranged and extend substantially vertically when the ventilator 1 is in an “upright” position.

As will be seen from FIG. 2, each elongate element 4 in the embodiment shown comprises a plurality of successive portions including a first portion 14 having the first end 4a, a second portion 34 having the second end 4b, and an intermediate portion 24 “sandwiched” between the first and second portions 14, 34, that is to say situated between these.

Preferably, the first and second end portions 14, 34 and the intermediate portion 24 are rectilinear or approximately rectilinear.

The first portion 14 forms a first angle A of greater than 90° with the intermediate portion 24, and the second portion 34 forms a second angle B of greater than 90° with the intermediate portion 24, said angles A, B being equal or different, for example angles of the order of 110 to 115°, for example of the order of 120 to 135°.

Moreover, as is illustrated in FIG. 1 and FIG. 2, the exoskeleton structure 3 is preferably configured to present four elongate elements 4 that are substantially vertical when the ventilator 1 is in the “upright” position.

Advantageously, the four elongate elements 4 each comprise an axis XX, which is for example carried by their intermediate portion 24, the different axes XX being parallel to each other.

Preferably, the exoskeleton structure 3 comprises two “rear” elongate elements 4 situated opposite the rear face 40 of the ventilator 1, that is to say the face opposite the front façade 10 with the screen 11, said two “rear” elongate elements 4 comprising coplanar bearing zones 44, which are situated for example in the intermediate portion 24 of axis AA, so as to be able to place the assembly composed of ventilator 1 and exoskeleton structure 3 in a “recumbent” position on a surface, for example the ground, as is illustrated in FIG. 5, such that the screen 11 of the ventilator 1 is located over said surface, that is to say substantially horizontally with respect to said surface, for example the ground or similar.

It should be noted that the exoskeleton structure 3 can also comprise additional strengthening elements (not shown), for example one or more intermediate wall elements joining two elongate elements 4 to each other, for example half way between the first and second ends 4a, 4b, so as to fix them firmly to each other and thereby further stiffen the exoskeleton structure 3.

As will be seen in FIG. 1 and in FIG. 3 to FIG. 5, the external housing 2 of the ventilator 1 comprises a front face or façade 10 supporting the screen 11, a rear face 40 (cf. FIG. 6), and two lateral faces, namely a right-hand lateral face 20 and a left-hand lateral face 30, which are arranged between the façade 10 and the rear face 40, on either side of the external housing 2. These four successive faces 10, 20, 30, 40 are separated by four corner regions 15 forming a join between the different faces 10, 20, 30, 40.

Advantageously, the elongate elements 4 of the exoskeleton structure 3 take up a position, wholly or in part, substantially opposite, i.e. facing and at a distance from, the corner regions 15, such as ridges, separating the façade 10 and the rear face 40 of the external housing 2 from the right-hand lateral face 20 and the left-hand lateral face 30.

Generally, the exoskeleton structure 3, in particular the base 6 and the elongate elements 4, in particular the two “rear” elongate elements 4 having coplanar bearing zones 44, is configured to ensure good vertical and horizontal stability of the ventilator 1 when it is placed either in an “upright” position as shown in FIG. 3 or in a “recumbent” position as shown in FIG. 5.

Moreover, as can be seen in FIG. 1 to FIG. 3 in particular, the exoskeleton structure 3 comprises four elongate elements arranged in pairs, in particular a pair of frontal elongate elements situated at the front face 10 of the ventilator 1, and the pair of rear elongate elements situated opposite the rear face 40 of the ventilator 1.

Advantageously, the two elongate elements 4 of a given pair are symmetrical with respect to each other and are additionally situated at an equal distance from the external housing of the ventilator 1. Thus, the right-hand frontal elongate element is symmetrical with the left-hand frontal elongate element and, similarly, the right-hand rear elongate element is symmetrical with the left-hand rear elongate element. Moreover, the two elongate elements 4 of two different pairs can also be symmetrical with one another.

According to a particular embodiment, the exoskeleton structure 3, in particular the bearing surface 16 of its base 6, can be configured in such a way that the ventilator is slightly inclined, and not strictly vertical, when it is placed in an “upright” position on a surface, in particular the ground, so as to improve the user's view of the HMI, in particular the screen 11, the selection keys or buttons, etc.

More generally, provision is made that the exoskeleton structure 3, which constitutes a gripping structure and also a protective structure situated around the ventilator 1, is designed such that no ventilator element protrudes from it, that is to say from the internal volume which it delimits and in which the ventilator 1 is inserted.

As has already been mentioned, the exoskeleton structure 3 allows the user of the assembly composed of ventilator 1 and exoskeleton structure 3 to grip it in several places via the main handle 9, situated above the ventilator 1, and via the elongate elements 4, thus making it easier to grip the assembly 1, 3 in different situations. The handle 9 is arranged at a sufficient distance 7 from the external housing 2 of the ventilator 1 to leave sufficient room for it to be taken hold of even when the user, for example a first responder, is wearing protective gloves, for example climbing gloves or fire-fighting gloves.

In addition, according to a particular embodiment, the exoskeleton structure 3 can be overmoulded with elastomer or the like in order to improve the impact resistance, for example in its lower part 6.

As is illustrated in FIG. 6, the exoskeleton structure 3 can comprise securing means 18, such as openings, loops, hooks or the like, which are configured to receive carabiners or other complementary fastening means, so as to allow the assembly composed of ventilator 1 and exoskeleton structure 3 to be secured in a vehicle, for example an ambulance, a helicopter or the like, or on a stretcher, a bed or the like.

In the embodiment shown in FIG. 6, the securing means 18 are openings formed in the joining zone 8 where the elongate elements 4 meet in pairs, on either side of the exoskeleton structure 3, thus forming the two Y-shaped or V-shaped joining structures 17 situated on either side of the central region that forms the gripping and carrying handle 9.

Generally, the exoskeleton structure 3 of the invention constitutes a rigid outer reinforcement of the ventilator 1, combining several functions and having many advantages, in particular.
protecting the ventilator 1 against droppage and impacts, thereby making the ventilator 1 more robust and reliable.
allowing the assembly to be gripped in several places, by virtue of the main gripping handle situated opposite the upper face or summit of the medical ventilator, and by virtue of the elongate elements 4 forming alternative gripping zones situated opposite the lateral faces of the ventilator 1.
permitting a good view of the HMI when the apparatus is placed on the ground, despite the presence of the exoskeleton structure 3.
ensuring the vertical and horizontal stability of the ventilator 1 when it is placed on the ground or on another surface, such as a table, the floor of a vehicle or similar.
protecting the ventilator 1 from the surface of the ground, thus avoiding direct contact between these, particularly when the ground is covered with or formed by sand, snow, soil or water, etc.
ensuring good cooling of the medical ventilator by having it raised off the ground.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A medical ventilator (1) comprising an external housing (2), and a rigid exoskeleton structure (3) arranged around said external housing (2) and rigidly connected to said external housing (2), the rigid exoskeleton structure (3) comprising a base (6) and a plurality of elongate elements (4) rigidly connected to said base (6) and defining a volume (5) in which the external housing (2) is housed by being positioned on the base (6), whereby the greater part of the rigid exoskeleton structure (3) is spaced (7) from said external housing (2) when said external housing (2) is housed in the volume (5), characterized in that the rigid exoskeleton structure (3) comprises at least one carrying handle (9) situated in a joining zone (8) of the elongate elements (4), allowing a user to manually grasp the exoskeleton structure (3) and transport the assembly (1, 3) composed of the medical ventilator and the exoskeleton structure.

2. The medical ventilator according to claim 1, characterized in that the elongate elements (4) are arranged substantially vertically from the base (6) when the ventilator (1) is in an upright position and resting on the base (6) of the exoskeleton structure (3).

3. The medical ventilator according to claim 2, characterized in that the elongate elements (4) comprise a first end rigidly connected to the base (6) and meet and join each other, via a second end, at a joining zone (8) of the exoskeleton structure (3) above the ventilator (1).

4. The medical ventilator according to claim 1, characterized in that the base (6) of the exoskeleton structure (3) comprises a bearing surface (16) and/or is curved outwards.

5. The medical ventilator according to claim 1, characterized in that the exoskeleton structure comprises four elongate elements (4).

6. The medical ventilator according to claim 5, characterized in that the four elongate elements (4) are arranged along the corner regions (15) of the ventilator (1) and are spaced apart from said regions.

7. The medical ventilator according to claim 5, characterized in that the elongate elements (4) have the general shape of a band or tape.

8. The medical ventilator according to claim 5, characterized in that, in the joining zone (8), the elongate elements (4) join each other in pairs, forming two Y-shaped or V-shaped joining structures (17) which are situated on either side of the exoskeleton structure (3) and are connected to each other by a central region that forms the carrying handle (9).

9. The medical ventilator according to claim 1, characterized in that the exoskeleton structure (3) comprises two rear elongate elements (4) situated opposite the rear face (40) of the external housing (2) of the ventilator (1), the two rear elongate elements (4) comprising coplanar bearing zones (44).

10. The medical ventilator according to claim 1, characterized in that the external housing (2) of the ventilator (1) comprises a front face (10) supporting a display screen (11) and/or a HMI, a rear face (40) situated opposite the front face (10), and two lateral faces (20, 30) arranged between the front face (10) and the rear face (40), said front face, rear face and two lateral faces being separated by four corner regions (15) forming a join between the faces (10, 20, 30, 40), and the one or more elongate elements (4) of the exoskeleton structure (3) being at least in part positioned opposite the corner regions (15) separating said faces (10, 20, 30, 40).

11. The medical ventilator according to claim 1, characterized in that the exoskeleton structure (3) forms a rigid cage that completely surrounds the external housing (2) of the ventilator (1).

12. The medical ventilator according to claim 1, characterized in that each elongate element (4) comprises a plurality of successive portions, including a first portion having the first end, a second portion having the second end, and an intermediate portion situated between the first and second portions, the first portion forming a first angle A of greater than 90° with the intermediate portion, and the second portion forming a second angle B of greater than 90° with the intermediate portion.

13. The medical ventilator according to claim 1, characterized in that the greater part of the exoskeleton structure (3) is spaced from the external housing (2) by a distance of less than 10 cm.

14. The medical ventilator according to claim 1, characterized in that the exoskeleton structure (3) is configured such that the ventilator (1) is slightly inclined with respect to the vertical when it rests in an upright position on a surface, particularly the ground.

15. The medical ventilator according to claim 10, characterized in that the exoskeleton structure (3) is moreover configured such that the ventilator (1) is slightly inclined when it rests in a recumbent position on a surface, and in that the screen (11) of the ventilator (1) is located at the top and in a horizontal position or in a position slightly inclined with respect to the horizontal position.
